(19) United States
(12) Patent Application Publication
(10) Pub. No.: US 2005/0079220 A1
(43) Pub. Date: Apr. 14, 2005

(54) Osmotic engine & dosage form for controlled release of a liquid active agent formulation

(76) Inventors: Betty Yu, Mountain View, CA (US); Liang-Chang Dong, Sunnyvale, CA (US); Andrew C. Lam, South San Francisco, CA (US); Crystal Pollock-Dove, Mountain View, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/902,912

(22) Filed: Jul. 30, 2004

Related U.S. Application Data
(60) Provisional application No. 60/492,002, filed on Jul. 31, 2003.

(51) Int. Cl. A61K 9/24

(52) U.S. Cl. 424/473

(57) ABSTRACT

The present invention relates to osmotic engines and dosage forms providing the controlled release of a liquid active agent formulation. More specifically, the present invention is directed to osmotic engines, dosage forms and methods that preserve osmotic engine functionality and reduce void volume formation in osmotically driven dosage form providing the controlled release of liquid active agent formulations.

### CROSS-REFERENCE TO RELATED APPLICATIONS

This application claims benefit of U.S. Provisional Patent application No. 60/492,002, filed Jul. 31, 2003, which is incorporated herein by reference.

### BACKGROUND

1. Field of the Invention

The present invention relates to osmotic engines and dosage forms providing the controlled release of a liquid active agent formulation. More specifically, the present invention is directed to osmotic engines, dosage forms and methods that preserve osmotic engine functionality and reduce void volume formation in osmotically driven dosage form providing the controlled release of liquid active agent formulations.

2. State of the Art

Osmotic dosage forms providing controlled release of liquid active agent formulations are known in the art. For example, U.S. Pat. No. 6,174,547 (“the ′547 Patent”), U.S. Pat. 5,830,502 (“the ′502 Patent”), U.S. Pat. No. 5,614,578 (“the ′578 Patent), International Publication Number WO 95/34285 (“the ′285 Publication”), and International Publication Number WO 01/41742 (“the ′742 Publication”) teach controlled release dosage forms configured to provide controlled release of liquid active agent formulations. The dosage forms taught in these references include a capsule that serves as a reservoir, a liquid active agent formulation contained within the capsule, an osmotic engine formed using an expandable osmotic composition positioned within the capsule, a rate controlling membrane formed over the capsule, and an exit orifice. As taught in the ′285 Publication, the expandable osmotic composition positioned within the capsule may be separated from the liquid active agent formulation by a barrier layer that is substantially impermeable to the passage of liquid. In operation, water from the environment of use is drawn into the expandable osmotic composition through the capsule wall. As water is drawn into the expandable osmotic composition, the osmotic engine expands within the capsule and expels the liquid active agent formulation into the environment of use through the exit orifice.

Although the dosage forms taught in the ′547 Patent, the ′502 Patent, the ′578 Patent, ′285 Publication, and the ′742 Publication are useful to achieve the controlled release of liquid active agent formulations, over time, the functionality of the osmotic engine included in such dosage forms may degrade. Where it occurs, degradation of the osmotic engine is evidenced by release rates that vary significantly from the targeted rate, by incomplete pumping of the liquid active agent formulation, or through detection of increased residual drug content in the osmotic engine. Moreover, where a dosage form is designed according to the teachings of the ′547 Patent, the ′502 Patent, the ′578 Patent, ′285 Publication, or the ′742 Publication, the amount of liquid active agent formulation available for release may be measurably reduced over time. In particular, void volumes may form within the reservoir containing the liquid active agent formulation. Such void volumes not only represent a depletion in the amount of liquid active agent formulation available for delivery from the dosage forms, but, where created, the void volumes can also interfere with or alter the release of the liquid active agent formulation, causing the release rate of active agent to vary away from a targeted value.

It has been found that the osmotic engine degradation and void volume formation exhibited in dosage forms manufactured according to the teachings of the ′547 Patent, the ′502 Patent, the ′578 Patent, the ′285 Publication, and the ′742 Publication may result from migration of the liquid active agent formulation into the expandable osmotic composition included in the osmotic engine. Even where such dosage forms include a barrier layer positioned between the expandable osmotic composition and the liquid active agent formulation, it has been found that the liquid active agent formulation included in the dosage forms may wick around the barrier layer and into the expandable osmotic composition. It has also been observed that, over time, liquid active agent formulation may even migrate into the barrier layer itself, which can further contributing to void volume formation within the dosage form and increase the amount of residual drug contained within the osmotic engine. If liquid active agent migrates into the expandable osmotic composition of the osmotic engine over time, the functionality of the osmotic engine may degrade. For example, where the liquid active agent formulation is hydrophobic, if it is absorbed into the expandable osmotic composition included in the osmotic engine, the hydrophobic formulation could prevent full hydration of the osmotic engine and cause a premature cessation of engine activity. In addition, as liquid active agent formulation migrates into the expandable osmotic composition, the amount of liquid active agent present within the reservoir of the dosage form decreases, resulting in void volume formation within the reservoir and a reduction in the amount of formulation available for delivery.

It would be an improvement in the art, therefore, to provide an osmotic engine that is not only useful in the production of dosage forms for the controlled delivery of a liquid active agent formulations, but is also designed to better resist permeation by the liquid active agent formulation included in the dosage forms. Such an osmotic engine would facilitate the manufacture of controlled-release, liquid active agent dosage forms that, over time, exhibit increased stability in release rate performance and a reduced occurrence of delivery or dosing problems that result from void volume formation. It will be apparent to those skilled in the art that a dosage form exhibiting these characteristics would further facilitate the development and commercialization of a dosage forms providing controlled delivery of active agents from liquid active agent formulations, particularly where prolonged storage of the dosage form is anticipated before administration.

### SUMMARY OF THE INVENTION

In one aspect, the present invention includes an osmotic engine suitable for use in dosage forms providing controlled delivery of active agent formulations. In particular, the present invention provides an osmotic engine that is resistant to permeation by liquid active agent formulations. In one embodiment, the permeation resistant osmotic engine of the present invention includes an expandable osmotic composition with a permeation resistant coating provided over at least a portion of the expandable osmotic composition. In another embodiment, the permeation resistant osmotic engine of the present invention includes an expandable osmotic composition encapsulated by a permeation resistant coating. Optionally, a permeation resistant osmotic engine according to the present invention may include a barrier layer. Where a permeation resistant engine according to the present invention includes both a barrier layer and a permeation resistant coating, the barrier layer may be provided within the permeation resistant coating or outside of the permeation resistant coating. The permeation resistant nature of the osmotic engine of the present invention facilitates the fabrication of dosage forms that not only provide controlled release of liquid active agent formulations, but also exhibit improved long-term release rate functionality and exhibit a reduced tendency to form void volumes within the dosage form over time.

Where the permeation resistant osmotic engine according to the present invention includes a permeation resistant coating, the configuration and formulation of the coating can change, depending on, for example, the nature of the liquid active agent formulation that may come in contact with the permeation resistant osmotic engine. In one embodiment, the permeation resistant coating is a hydrophobic coating formulated to reduce or prevent permeation by an aqueous or otherwise hydrophilic liquid active agent formulation. In another embodiment, the permeation resistant coating is a hydrophilic coating formulated to reduce or prevent permeation by a hydrophobic liquid active agent formulation. However, a permeation resistant coating included in a permeation resistant osmotic engine according to the present invention does not adversely affect the release rate performance provided by the engine. Therefore, where the permeation resistant osmotic engine of the present invention includes a permeation resistant coating, the coating is formulated or configured to allow the passage of water from an environment of operation into the expandable osmotic composition included in the permeation resistant osmotic engine.

In another aspect, the present invention includes a method for manufacturing a permeation resistant osmotic engine. In one embodiment, the method according to the present invention for manufacturing a permeation resistant engine includes providing an expandable osmotic composition and coating said expandable osmotic composition with a permeation resistant coating the covers at least a part of an outside surface of the expandable osmotic composition. In another embodiment, the method according to the present invention for manufacturing a permeation resistant engine includes substantially encapsulating an expandable osmotic composition in a permeation resistant coating. In yet another embodiment of the method according to the present invention for manufacturing a permeation resistant engine, the method includes providing an expandable osmotic composition and a barrier layer and providing a permeation resistant coating over an outside surface of the barrier layer and over at least a portion of the outside surface of the expandable osmotic composition. In such an embodiment, the expandable osmotic composition and barrier layer can be provided as a bi-layer tableted composition. In yet another embodiment, the method of the present invention for fabricating a permeation resistant engine includes providing an expandable osmotic composition, coating said composition with a permeation resistant coating that at least partially covers an outside surface of the expandable osmotic composition, followed by positioning a barrier layer over an outside surface of the permeation resistant coating. Where the barrier layer is provided on an outside surface of the permeation resistant coating, the barrier layer may be adhered to or simply positioned in contact with the permeation resistant coating.

In another aspect, the present invention includes an osmotic dosage form that provides controlled delivery of a liquid active agent formulation. A dosage form of the present invention includes a permeation resistant osmotic engine according to the present invention, a reservoir, and a liquid active agent formulation contained within the reservoir. A dosage form according to the present invention is configured to provide the controlled release of the liquid active agent formulation, and the design of a dosage form according to the present invention, works to reduce or prevent migration of the liquid active agent formulation into the expandable osmotic composition included in the osmotic engine. In particular, the permeation resistant osmotic engine included in a dosage form according to the present invention serves to reduce, or prevent altogether, migration of the liquid active agent formulation into the expandable osmotic composition included in the permeation resistant osmotic engine. The design of a dosage form according to the present invention, therefore, works to better preserve the release rate functionality of the dosage form over time reduces the occurrence of void volume formation within the reservoir of the dosage form.

In one embodiment, the dosage form of the present invention includes a reservoir, a liquid active agent formulation within the reservoir, a permeation resistant osmotic engine that includes an expandable osmotic composition and a permeation resistant coating, a rate controlling membrane, and an exit orifice through which the liquid active agent formulation can be delivered. The rate controlling membrane is configured and formulated such that, upon administration of the dosage form to an environment of operation, water passes into the expandable osmotic composition through the rate controlling membrane at a controlled rate. The controlled influx of water into the expandable osmotic composition of the permeation resistant osmotic engine causes the expandable osmotic composition to expand, resulting in the controlled expulsion of the liquid active agent formulation through the exit orifice.

In yet another aspect, the present invention includes a method for fabricating a dosage form providing controlled release of a liquid active agent formulation. In each embodiment, the method of the present invention for fabricating a controlled-release dosage form includes providing a permeation resistant engine, a reservoir, and a liquid active agent formulation, loading the liquid active agent formulation into the reservoir, and operatively associating the permeation resistant engine, the reservoir and the liquid active agent formulation such that, as the permeation resistant engine operates, liquid active agent formulation is expelled from the reservoir. Any embodiment of a permeation resistant osmotic engine according to the present invention may be used in the method for fabricating a controlled release dosage form. The method of the present invention for fabricating a dosage form providing controlled release of a liquid active agent formulation also includes providing a rate controlling membrane that is formulated and configured to provide controlled expansion of the permeation resistant engine upon administration of the dosage form to an environment of operation and providing an exit orifice that allows the liquid active agent formulation to be expelled from within the reservoir as the dosage form operates.

### BRIEF DESCRIPTION OF THE DRAWINGS

FIG. 1 through FIG. 8 provide cross-sectional representations of permeation resistant osmotic engines and dosage forms according to the present invention.

FIG. 9 provides a graphical representation of liquid formulation uptake by permeation resistant engines representing one embodiment of the permeation resistant engine of the present invention compared to the liquid formulation uptake of osmotic engines that were not prepared according to the present invention.

FIG. 10 provides a graphical representation of the release rate functionality of two different embodiments of the dosage form of the present invention compared to the release rate functionality of two other dosage forms prepared without permeation resistant engines.

FIG. 11 provides the cumulative release rate performance of two different embodiments of the dosage form of the present invention compared to the cumulative release rate performance of two other dosage forms prepared without permeation resistant engines.

### DETAILED DESCRIPTION OF THE INVENTION

The present invention includes a permeation resistant osmotic engine. A permeation resistant osmotic engine according to the present invention includes an expandable osmotic composition. The terms “permeation resistant osmotic engine” and “permeation resistant engine” are used interchangeably herein and indicate an osmotic engine that includes an expandable osmotic composition and is configured or formulated such that, when included in a dosage form, the osmotic engine exhibits an uptake of liquid active agent formulation that is less than 5% by weight before administration of the dosage form. Preferably, the permeation resistant osmotic engine of the present invention is formulated or configured such that, when included in a dosage form, the osmotic engine exhibits an uptake of liquid active agent formulation of 3% by weight, or less, before administration of the dosage form, with permeation resistant engines exhibiting liquid active agent formulation uptake of 1% by weight, or less, before administration of the dosage form being particularly preferred. The permeation resistant osmotic engine according to the present invention reduces or prevents the potential performance problems associated with the migration of liquid active agent formulation to be delivered from a dosage form into the expandable osmotic composition included in the osmotic engine.

An expandable osmotic composition included in a permeation resistant engine of a dosage form according to the present invention may be formulated and formed using any materials and means that result in a composition that can be operatively associated with the reservoir included in a dosage form, is acceptable for the intended application of the dosage form, exhibits sufficient osmotic pressure to draw in water from an environment of use over a desired period of time, and expands to exert a force sufficient to cause expulsion of a liquid active agent formulation from within a reservoir as water is taken into the composition. The expandable osmotic composition included in a permeation resistant engine according to the present invention can be manufactured using known materials and methods, and may be formulated to provide an expandable osmotic composition that is itself permeation resistant or can be made permeation resistant. Presently, the expandable osmotic composition included in a permeation resistant engine according to the present invention is preferably formed as a tableted composition that includes a hydrophilic polymer capable of swelling or expanding upon interaction with water or aqueous biological fluids.

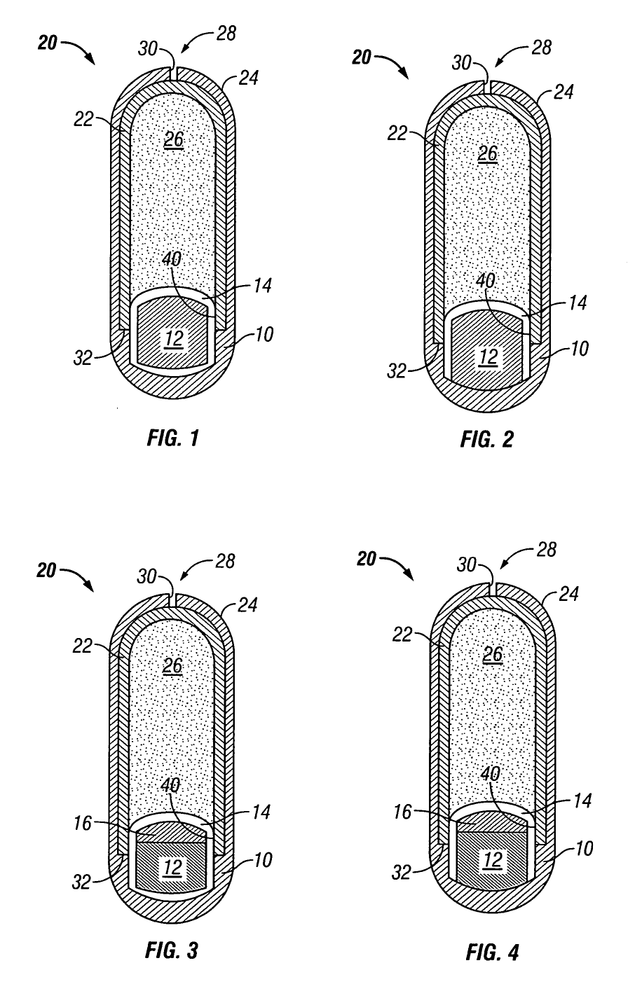

The expandable osmotic composition included in a permeation resistant engine according to the present invention may further include an osmagent to increase the osmotic pressure exerted by the expandable osmotic composition, a suspending agent to provide stability and homogeneity to the expandable osmotic composition, a tableting lubricant, an antioxidant, or a non-toxic colorant or dye. Materials and methods that can be used to form an expandable osmotic composition suitable for use in a permeation resistant osmotic engine of the present invention are taught, for example, in U.S. Pat. Nos. 6,174,547 and 6,245,357 and in patent publications numbered WO 95/34285, US-2002-0071863 A1, US-2003-0232078 A1, and US-2003-0918619 A1, the contents of each of which are herein incorporated in their entirety by reference.

Where the expandable osmotic composition included in an osmotic engine according to the present invention is formed of a tableted, hydrophilic polymer composition, the expandable osmotic composition will typically require further processing in order to render the expandable osmotic composition resistant to permeation by a liquid active agent formulation. For example, the expandable osmotic composition may be provided with a permeation resistant coating over at least an area of the expandable osmotic composition, wherein the coating is formulated to be resistant to permeation by a given liquid active agent formulation. Therefore, as is illustrated in FIG. 1, one embodiment of a permeation resistant osmotic engine 10 according to the present invention includes an expandable osmotic composition 12 covered by a permeation resistant coating 14.

The materials used to form a permeation resistant coating 14 included in a permeation resistant osmotic engine 10 of the present invention will vary depending on the nature of the liquid active agent formulation to which the expandable osmotic composition must be made permeation resistant. In particular, to render the expandable osmotic composition 12 resistant to permeation by a hydrophobic liquid active agent formulation, a permeation resistant coating 14 provided over the expandable osmotic composition will typically be a hydrophilic coating that is substantially impermeable to the hydrophobic liquid active agent formulation. Alternatively, to render the expandable osmotic composition 12 resistant to permeation by a hydrophilic liquid active agent formulation, a permeation resistant coating 14 provided over the expandable osmotic composition will typically be a hydrophobic coating that is substantially impermeable to the hydrophilic liquid active agent formulation. As used herein, “substantially impermeable” refers to a coating composition that is sufficiently impermeable to a liquid active agent formulation to render the expandable osmotic composition permeation resistant as defined herein. In each case however, a permeation resistant coating 14 included in a permeation resistant osmotic engine 10 according to the present invention is formulated and configured to allow the expandable osmotic composition 12 included in a permeation resistant engine 10 to function as necessary when the permeation resistant engine 10 is incorporated into a dosage form.

A permeation resistant coating 14 may be formulated using a variety of different naturally derived or synthetic materials. Examples of materials that may be used in formulating a permeation resistant coating 14 that is substantially impermeable to a hydrophobic liquid active agent formulation include, but are not limited to, naturally derived animal materials, such as albumin animal glue, casein, shellac, beeswax, naturally derived plant materials, such as oils, resins, waxes, rubbers, gum Arabic, tragacanth, colophony, balsam, carnauba wax, linseed oil, and plant-derived proteins, starches, and dextrins, inorganic and mineral materials, such as silicates, magnesia, phosphates, litharge, and sulfur containing materials, synthetically derived materials, such as synthetic elastomers, synthetic rubbers, butyl, polisobutylene, polybutadiene blends, polyisoprenes, polychloroprene, polyurethane, silicone, polysulfide, and polyolefins, thermoplastic materials and cellulose derivatives, such as acetate, acetate-butyrate, caprate, nitrate, methyl cellulose, hydroxyl ethyl cellulose, ethyl cellulose, carboxy methyl cellulose, vinyl polymers, such as polyvinyl acetate, polyvinyl alcohol, and polyvinyl chloride, polyester materials, such as polyesters, polystyrenes, and polyamides, polyacrylate materials, such as methacrylate and acrylate polymers, cyanoacrylates, polyether materials, such as polyhydroxyether and polyphenolic ethers, polysulfone materials, thermosetting amino plastics, such as urea and melamine formaldehydes, epoxy materials, such as epoxy polyamide, epoxy bitumen, epoxy polysulfide, and epoxy nylon, phenolic resins, such as phenol and resorcinol formaldehydes, phenolic-nitrile, phenolic-neoprene, and phenolic-epoxy, polyaromatic materials, such as polyimides, polybenzimidazole, and polyphenylene, and furane materials, such as phenol furfural. Presently, hydrophilic polymer materials, such as hydroxypropylmethylcellulose (HPMC) and hydroxyethylcellulose (HEC), are preferably used to form permeation resistant coatings 14 that are substantially impermeable to hydrophobic liquid active agent formulations. Examples of materials that may be used to provide a permeation resistant coating 14 that is substantially impermeable to a hydrophilic liquid active agent formulation include, but are not limited to, latex materials. For example, Surelease® latex materials, which are available from Colorcon, Inc., Kollicoat® SR latex materials, which are available from BASF, Eudragit® SR, and other polymethylacrylate latex materials are can be used to provide a permeation resistant coating 14 that is substantially impermeable to a hydrophilic liquid active agent formulation.

Where desired, a permeation resistant coating 14 may be formulated using blends of materials that provide desirable coating characteristics. For example, in order to achieve a permeation resistant coating 14 having desirable coating characteristics, it may be necessary to formulate the coating material using blends of film forming materials. In addition, a permeation resistant coating 14 according to the present invention may include one materials, such as a plasticizer, that improve the coating characteristics provided by a film forming material or a blend of film forming materials. In particular, where HPMC is used to form a permeation resistant coating 14 included in a permeation resistant engine according to the present invention, it is presently preferred that the HPMC coating is formulated using a plasticizer, such as PEG 8000. Importantly, a permeation resistant coating 14 is preferably formulated such that tensile strength of the permeation resistant coating 14 can be overcome by the force exerted by the expandable osmotic composition 12 as the permeation resistant engine 14 functions and the expandable osmotic composition expands 12.

As can be seen in FIG. 1, where the permeation resistant coating 14 provided over the expandable osmotic composition 12 is permeable to the passage of water, such as a coating that includes a hydrophilic polymer or water soluble component, the permeation resistant coating 14 may completely encapsulate the expandable osmotic composition 12. A permeation resistant coating 14 that encapsulates the expandable osmotic composition 12 is formulated to exhibit a water permeability that is sufficient to permit water to enter the expandable osmotic composition 12 at a rate that allows the permeation resistant engine 10 to expand as needed to provide a desired release rate of active agent formulation. Moreover, if desired, the thickness and water permeability of a permeation resistant coating 14 that encapsulates an expandable osmotic composition 12 may be adjusted to provide a further measure of control over the release characteristics of a dosage form incorporating a permeation resistant engine 10 according to the present invention. For example, in order to delay delivery of a liquid active agent formulation from a dosage form that incorporates a permeation resistant engine 10 having a permeation resistant coating 14 that encapsulates the expandable osmotic composition 12 and is permeable to water, the thickness of permeation resistant coating 14 may be increased until a desired delay is achieved.

However, as is shown in FIG. 2, a permeation resistant coating 14 included in a permeation resistant engine 10 according to the present invention need not entirely encapsulate the expandable osmotic composition 12. In fact, where a permeation resistant coating 14 included in a permeation resistant engine 10 according to the present invention is impermeable to water or is not sufficiently permeable to water to allow the permeation resistant engine 10 to function as desired, the permeation resistant coating 14 is configured such that the permeation resistant coating 14 does not encapsulate the expandable osmotic composition 12. In that manner, the water can be taken up by the expandable osmotic composition 12 at a rate that enables the permeation resistant engine 10 to function as desired.

In some instances, the permeation resistant nature of a permeation resistant engine 10 according to the present invention may eliminate the need to include an additional barrier layer in the permeation resistant engine 10. Where such is the case, a permeation resistant engine 10 of the present invention works to not only preserve engine functionality, but also to increase drug loading of a dosage form incorporating the engine, as the amount of liquid active agent formulation included in the dosage form can be increased by the volume normally occupied by the barrier layer.

Nevertheless, a permeation resistant engine 10 according to the present invention may also include a barrier layer 16, as is shown in FIG. 3 through FIG. 8. When the permeation resistant engine 10 is associated with a controlled release dosage form, the use of a barrier layer 16 may further reduce or prevent the mixing of active agent formulation included in the dosage form with the expandable osmotic composition 12 included in the permeation resistant engine 10, particularly after the dosage form is administered to an environment of operation and the permeation resistant engine 10 functions within the dosage form. Therefore, although a barrier layer 16 may not be necessary, the use of a barrier layer 16 in a permeation resistant engine 10 according to the present invention can work to further reduce the amount of residual active agent that remains within a dosage form after the permeation resistant engine 10 has ceased to function or has filled the interior of a reservoir included in a dosage form. The barrier layer 16 also serves to increase the uniformity with which the driving power of the expandable osmotic composition 12 is transferred an active agent formulation to be delivered from a dosage form.

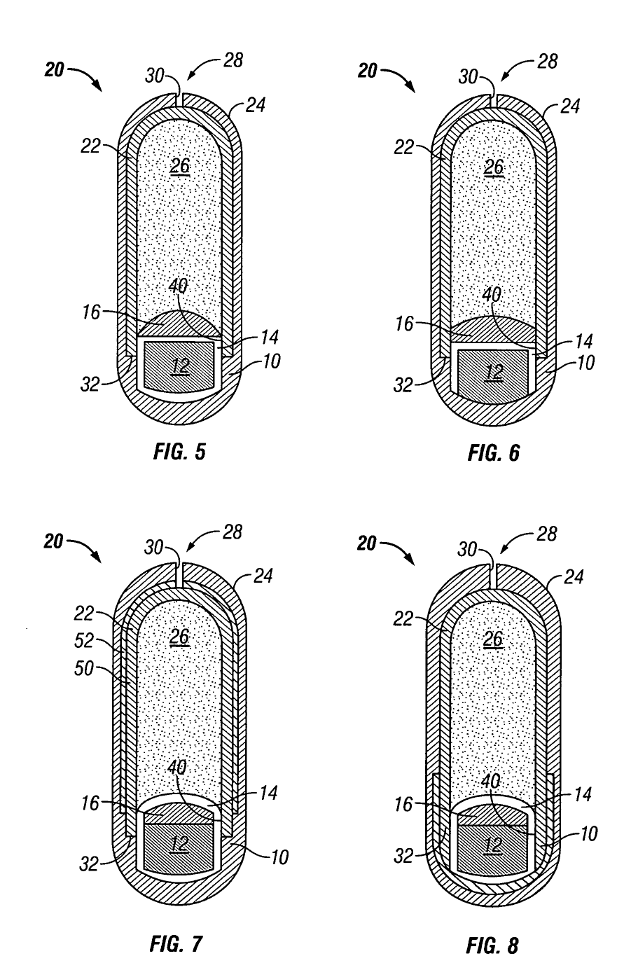

A barrier layer 16 included in a permeation resistant osmotic engine 10 according to the present invention is formulated to of composition that is substantially impermeable to liquid compositions. Materials suitable for forming a barrier layer 16 useful in an permeation resistant engine 10 according to the present invention include, but are not limited to, a polymeric composition, a high density polyethylene, a wax, a rubber, a styrene butadiene, a calcium phosphate, a polysilicone, a nylon, Teflon®, a polystyrene, a polytetrafluoroethylene, halogenated polymers, a blend of a microcrystalline, high acetyl cellulose, or a high molecular weight fluid impermeable polymer.

Where a permeation resistant engine 10 according to the presenting invention includes a barrier layer 16 and a permeation resistant coating 14, the barrier layer 16 may be provided within the permeation resistant coating 14, as is shown in FIG. 3 and FIG. 4, or on an outside surface of the permeation resistant coating 14, as is shown in FIG. 5 and FIG. 6. Fabricating a permeation resistant engine 10 according to the present invention with a barrier layer 16 that is in direct contact with the expandable osmotic composition 12 and is positioned within the a permeation resistant coating 14, allows the expandable osmotic composition 12 and barrier layer 16 to be formed as a tableted, bi-layer composition, which can then be coated with a permeation resistant coating 14. Materials and methods suitable for creating a bi-layer tablet including an expandable osmotic composition 12 and a barrier layer 16 are taught, for example, in patent publications numbered WO 95/34285, US-2003-0232078 A1, and US-2003-0198619 A1, the contents of each of which are incorporated in their entirety herein by reference.

A barrier layer 16 included on the outside surface of a permeation resistant engine 10 included a permeation resistant coating 14 can be positioned on the outside surface of the permeation resistant coating 14 using any suitable method. For example, the barrier layer 16 may be formed as desired, such as by a suitable tableting technique, and then adhered to the outside surface of the permeation resistant coating 14 using any suitable adhesive material or technique. Alternatively, a barrier layer 16 positioned outside a permeation resistant coating 14 need not be adhered to or form part of the permeation resistant engine 10 until the engine is positioned within a dosage form, at which point the dosage form may be assembled such that the permeation resistant engine 10 includes a barrier layer in contact with the permeation resistant coating 14 and positioned between the drug formulation and the expandable osmotic composition included 12 in the permeation resistant engine 10.

A permeation resistant coating 14 included in a permeation resistant engine 10 according to the present invention can be created using any method that provides a permeation resistant coating of a desired configuration and thickness over the expandable osmotic composition 12 included in the permeation resistant engine 10. For example, a permeation resistant coating 14 may be provided over the expandable osmotic composition 12 using spray coating or dip coating technologies known in the art. Alternatively, a permeation resistant coating 14 may be provided over the expandable osmotic composition using a shrink-wrapping process that includes coating the expandable osmotic composition in, for example, a shape-memory polymer material, and processing the polymer material such that is shrinks to fit and form a permeation resistant coating 14.

The present invention also includes a dosage form that provides controlled-release of a liquid active agent formulation. A dosage form according to the present invention includes a reservoir, a liquid active agent formulation included in the reservoir, a permeation resistant engine according to the present invention, and an exit orifice. The permeation resistant engine is positioned within the dosage form such that, as the engine functions, the expandable osmotic composition included in the permeation resistant engine expands into the reservoir and expels the liquid active agent from within the reservoir through the exit orifice. A dosage form according to the present invention is also configured such that, upon administration of the dosage form to an environment of operation, water is taken up by the expandable osmotic composition included in the permeation resistant engine at a controlled rate, resulting in controlled expansion of the permeation resistant engine. The controlled expansion of the permeation resistant engine, in turn, effects the controlled expulsion or release of liquid active agent formulation from the dosage form.

FIG. 1 through FIG. 8 illustrate various embodiments of the dosage form of the present invention. Each of the embodiments of the dosage form 20 illustrated in FIG. 1 through FIG. 8 include a permeation resistant engine 10 that is made resistant to permeation by a liquid active agent formulation 26 by coating the expandable osmotic composition 12 with a permeation resistant coating 14. As can be seen in FIG. 1 through FIG. 7, a dosage form 20 of the present invention is preferably configured such that the permeation resistant osmotic engine 10 is positioned only partially within the reservoir 22. In the embodiments illustrated in the figures, the dosage form 10 also includes a rate controlling membrane 24, which, in operation, works to control the rate at which water enters the expandable osmotic composition 12 included in permeation resistant engine 10. Therefore, the rate controlling membrane 24 included in the embodiments illustrated in FIG. 1 through FIG. 8 facilitates the controlled expansion of the permeation resistant engine 10 into the reservoir 22, which results in expulsion of the liquid active agent formulation 26 through the exit orifice 28 at a controlled rate.

The reservoir 22 included in a dosage form 20 of the present invention is formed to contain a desired amount of liquid active agent formulation 26 and may be formed as desired to accommodate one or more components of a controlled release dosage form 20 of the present invention. For example, the reservoir 22 can be formed with a first end 32 that includes an opening 40 that is sized and shaped to accommodate a permeation resistant engine 10. Moreover, though the reservoir 22 of a dosage form 10 of the present invention may be formed in a generally oblong shape, the dosage form 20 according to the present invention is not so limited and may be manufactured to include a reservoir 22 that is sized and shaped as desired to suit a particular dosage form or active agent delivery application.

Though the reservoir may be formed as a capsule that completely surrounds the permeation resistant osmotic engine 10, as is shown in FIG. 8, it is presently preferred that a dosage form 20 of the present invention include a reservoir 22 that does not completely enclose the permeation resistant engine 10. Designing the dosage form 20 such that the reservoir 22 does not completely enclose the permeation resistant engine 10 simplifies the dosage form and works to improve long-term structural stability of the dosage form. The high level of osmotic activity of the expandable osmotic compositions useful in a permeation resistant osmotic engine may dehydrate a capsule or reservoir forming material that encloses the permeation resistant engine to such a degree that the capsule or reservoir material becomes brittle and cracks, or is otherwise structurally compromised, before administration. The designs of the dosage forms illustrated in FIG. 1 through FIG. 7 allow any interaction between the reservoir forming material and the expandable osmotic composition 12 included in the permeation resistant engine 10 to be minimized or avoided altogether until after the dosage form is administered and begins operation. In this manner, it is believed that the designs illustrated in FIG. 1 through FIG. 7 not only simplify the design of a dosage form 20 according to the present invention, but serve to improve the structural stability of the dosage form 20 over time.

Designing a dosage form 20 according to the present invention to include a reservoir 22 that does not entirely enclose the permeation resistant engine 10 also facilitates the use of water impermeable reservoir materials where desired. The proper function of permeation resistant engine 10 according to the present invention depends on an influx of water from an environment of operation, and if the reservoir 22 is formed of a water impermeable material and is configured such that the reservoir 22 completely encloses the permeation resistant engine 10, the permeation resistant engine 10 could not function as desired to provide the controlled release of a liquid active agent formulation.

The reservoir 22 included in an oral dosage form 10 of the present invention may be formed of a variety of materials. Any material that is compatible with a desired liquid active agent formulation, is capable of being formed into a reservoir of desired shape and size, is suitable for use in an oral dosage form, and is capable of withstanding the anticipated storage conditions and operational stresses can be used to provide the reservoir 22 included in a dosage form 20 according to the present invention. Depending on the liquid active agent formulation 26 included in the dosage form 20 and the desired performance characteristics of the dosage form 20, the reservoir may be formed of a water permeable or water impermeable material. A reservoir 22 useful in a dosage form according to the present invention may be fabricated by any suitable method. Examples of materials and methods, such as suitable dip coating and injection molding techniques are described in, for example, U.S. Pat. Nos. 6,183,466, 6,174,547, 6,153,678, 5,830,502, and 5,614,578, in patent publications numbered WO 95/34285, US-2002-0071863 A1, US 2004 0058000 A1, US-2003-0232078 A1, and US-2003-0198619 A1, the contents of each of which are incorporated by reference herein in their entirety.

Water permeable materials that may be used to form a reservoir 22 included in a dosage form 20 of the present invention include materials typically used to fabricate orally deliverable, liquid filled capsules. For example, a water permeable reservoir 22 included in a dosage form 20 of the present invention may be formed using hydrophilic polymer materials or hydrophilic gelatin materials, such as hydrophilic gelatin materials commonly used to form orally administrable capsules. Hydrophilic polymer materials, including cellulosic materials, provide preferred water permeable materials that may be used to form a reservoir 22 useful in a dosage form 20 of the present invention. Relative to the gelatin materials that are typically used in dosage form fabrication, water-soluble polymer materials are less susceptible to moisture loss and are less sensitive to changes in moisture content. As a result, a reservoir 22 formed using a hydrophilic polymer material is typically better able to retain its structural integrity upon exposure to the liquid active agent formulation 26 and the permeation resistant engine 10 included in a dosage form 20 of the present invention. Moreover, because hydrophilic polymer materials are generally less susceptible to moisture loss, a reservoir 22 manufactured using hydrophilic polymer materials can be made such that less water is available to be drawn into the liquid active agent formulation 26 from within the materials forming the reservoir 22 itself.

Where a reservoir 22 of a dosage form 20 of the present invention is formed using a water permeable material, it is presently preferred that the water permeable material be formed of a hydrophilic polymer material. The structural stability of gelatin materials, such as the gelatin materials typically used to create capsules for the delivery of liquid formulations, is sensitive to changes in hydration. In particular, it has been found that typical gelatin materials become brittle and may crack if moisture content drops below about 8%. However, if the moisture content of typical gelatin materials exceeds about 13%, the material can become too soft and tacky for further processing steps, such the process steps necessary to provide the reservoir with one or more desired coatings or subcoats. Such sensitivity to moisture content is problematic because the liquid active agent formulation 26 and the expandable osmotic composition 12 included in a permeation resistant osmotic engine 10 can exhibit relatively high osmotic activity, which can cause water to migrate out of a gelatin material to such a degree that the material becomes brittle, cracks, or is rendered structurally unsuitable. Therefore, even though gelatin materials may be used to provide a reservoir 22 of a dosage form 20 of the present invention, such materials are not presently preferred, particularly where liquid active agent formulation 26 included in the dosage form exhibits a relatively high osmotic activity and it is desired that the dosage form have an extended shelf life.

Hydrophilic polymer materials that may be used to as the water permeable material included in a multilayer reservoir 22 include, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water permeable material included in a reservoir 22 of a dosage form 20 of the present invention may be manufactured using a single polymer material, the water permeable material may also be formed using a mixture of more than one polymer. Presently, because HPMC capsules for oral delivery of liquid active agent formulations are commercially available and it has been found that capsule bodies formed of HPMC can be used to provide a reservoir 22 exhibiting suitable performance characteristics, the water permeable material included in a reservoir 22 of a dosage form 20 of the present invention is preferably formed using an HPMC material.

Where the reservoir 22 is formed of a material that is impermeable to water, the reservoir 22 can be made using a single material or a combination of materials. The material used to create a reservoir 22 that is suitable for use in a dosage form 20 according to the present invention and is impermeable to water according to the present invention need not be perfectly impermeable to the passage of water. As it is used herein, the term “impermeable” refers to reservoir formed of a material that exhibits a water flux of less than about 10⁻⁴ (mil·cm/atm·hr). Where the reservoir 22 included in a dosage form of the present invention is formed using a water impermeable material, the water impermeable nature of the material serves to reduce or prevent migration of water from an external environment, through the reservoir, and into the liquid active agent formulation.

In one embodiment, a water impermeable reservoir 22 suitable for use in a dosage form 20 according to the present invention is formed using a single layer of material that is impermeable to the passage of water. Materials suitable for forming such a reservoir 22 include, but are not limited to, water impermeable polymer materials. Where a single layer of water impermeable polymer material is used to form the reservoir 22, the polymer is preferably a synthetic resin or a combination of synthetic resins. Examples of water impermeable synthetic resins that may be used to form the reservoir 22 include, for example, linear polycondensation resins, condensation polymerized resins, addition polymerized resins, resins of phthalic anhydrides, polyvinyl resins such as polyethylene, polypropylene and their copolymers, polymer resins of methacrylic acid esters and acrylic acid esters, polycaprolactone, and copolymers of polycaprolactone with dilactide, diglycolide, valerolactone or decalactone. Different impermeable polymer materials and different combinations of impermeable polymer materials may be chosen to provide a reservoir 12 providing desired permeability, compatibility, and stability characteristics. A water impermeable reservoir may be formed, for example, using coating or molding techniques that are known in the art, such as, for example, those techniques described in U.S. Pat. Nos. 6,183,466, 6,153,678, 5,830,502, and 5,614,578 and in U.S. patent publication numbered US-2004-0058000 A1.

In an alternative embodiment, a water impermeable reservoir 22 included in a dosage form 20 according to the present invention may include two or more layers of different materials. For example, as is illustrated in FIG. 7, a reservoir 22 of a dosage form 20 of the present invention can include a water permeable material 50 coated with a water impermeable subcoat 52. The water permeable material 50 may be formed of a substance that is hydrophilic or otherwise permeable to the passage of water, such as the hydrophilic polymer and gelatin materials already described herein. The water permeable material 50 included in a water impermeable reservoir 22 included in a dosage form 20 according to the present invention may also be formed of a combination of water permeable and water impermeable materials. The water permeable material included in such a reservoir 22 may be formulated and formed by known methods, such as by the techniques described herein as useful in forming a water permeable reservoir 22 formed of a hydrophilic polymer or gelatin material. A water impermeable subcoat 16 included in a reservoir 22 of a dosage form 20 according to the present invention may be formed using any suitable water impermeable material that can be coated on or otherwise provided over the water permeable material 50. However, latex materials, such as Surelease® latex materials, which are available from Colorcon, Inc., Kollicoat® SR latex materials, which are available from BASF, Eudragit® SR, and other polymethylacrylate latex materials, are presently preferred for forming a water impermeable subcoat 16. A water impermeable subcoat 52 may be provided over the water permeable material 50 included in a water impermeable reservoir 22 of a dosage form according to the present invention using any suitable coating or lamination technique. Coating processes suitable for providing a water impermeable subcoat 52 are described, for example, in U.S. patent publication numbered US-2004-0058000 A1.

A rate controlling membrane 24 included on a dosage form 20 of the present invention allows water or aqueous fluid to enter the permeation resistant osmotic engine at a controlled rate and thereby facilitates controlled expansion of the permeation resistant engine 10. A rate controlling membrane 24 included in a dosage form 20 according to the present invention is non-toxic in the intended environment of operation and maintains its physical and chemical integrity during the operation of the dosage form 20. Adjusting the thickness or chemical make-up of the rate controlling membrane 24 can control the rate at which the expandable osmotic composition 12 included in a permeation resistant engine 10 expands after the dosage form 20 is administered. Therefore, a rate controlling membrane 24 included in an oral dosage form 10 of the present invention serves to control the release rate or release rate profile achieved by a dosage form 20 according to the present invention.

A rate controlling membrane 24 for use in a dosage form 10 of the present invention may be formed using any material that is permeable to water, is substantially impermeable to the active agent, is pharmaceutically acceptable, and is compatible with the other components of the dosage form of the present invention. Generally, a rate controlling membrane 24 will be formed as a semipermeable membrane using materials that include semipermeable polymers, semipermeable homopolymers, semipermeable copolymers, and semipermeable terpolymers. Semipermeable polymers are known in the art, as exemplified by U.S. Pat. No. 4,077,407, which is incorporated herein by this reference, and they can be made by procedures described in Encyclopedia of Polymer Science and Technology, Vol. 3, pages 325 to 354, 1964, published by lnterscience Publishers, Inc., New York. A rate controlling membrane 24 included in the dosage form 10 of the present invention may also include a plasticizer to impart flexibility and elongation properties to the rate controlling membrane 24 or a flux regulating agent, such as a flux enhancing or a flux reducing agent, to assist in regulating the fluid permeability or flux through the rate controlling membrane 24.

A rate controlling membrane 24 included in a dosage form 20 according to the present invention is provided over at least the portion of the reservoir 22 or permeation resistant engine 10 included in a dosage form 20 according to the present invention. Where the dosage form in includes a reservoir 22 that encapsulates the permeation resistant engine, as illustrated in FIG. 8, a rate controlling membrane 24 is provided over at least a portion of the reservoir in a manner that results in the controlled hydration of the permeation resistant engine 10 upon administration of the dosage form 20. Where the dosage form 20 of the present invention is configured such that the permeation resistant engine 10 is only partially inserted into the reservoir 22 and is not completely encapsulated by the reservoir 22, a rate controlling membrane 24 included in the dosage form is provided over at least the portion of the permeation resistant engine 10 that is not enclosed within the reservoir 22. However, as is shown in FIG. 1 through FIG. 7, a rate controlling membrane 24 included in a dosage form 10 of the present invention may also be provided over both the reservoir 22 and an exposed portion of the permeation resistant engine 10. Where a dosage form 20 according to the present invention includes a reservoir 22 that is permeable to water, a controlling membrane 24 included in the dosage form 20 preferable extends over both the reservoir 22 and any exposed portion of the permeation resistant osmotic engine 10.

Methods for providing a rate controlling membrane 24 suitable for use in a dosage form 20 according to the present invention are known in the art and include any suitable coating technique, such as a suitable dip coating or spray coating process. Additional references describing materials and methods suitable for fabricating rate controlling membranes suitable for use in a oral dosage form 20 of the present invention include, for example, U.S. Pat. Nos. 6,174,547 and 6,245,357 and patent publications numbered WO 95/34285, US-2002-0071863 A1, US-2003-0232078 A1, and US-2003-0198619 A1, the contents which are incorporated in their entirety herein by reference.

The dosage form 20 of the present invention may be provided with any desired liquid active agent formulation 26. As it used herein, the expression “active agent” encompasses any drug, therapeutic compound, or composition that can be delivered to provide a benefit to an intended subject. The expression “liquid active agent formulation” is used herein to indicate a formulation that contains an active agent and is able to flow from a dosage form 20 of the present invention into an environment of use. A liquid active agent formulation 26 suitable for use in the dosage form of the present invention may be neat liquid active agent or a solution, suspension, slurry, emulsion, self-emulsifying composition, liposomal composition, or other flowable formulation in which the active agent is present. The liquid active agent formulation 26 may be a solid, or not flowable, at temperatures lower than the temperature of the desired operational environment, such as the body temperature of an intended animal or human subject, but such a formulation should become flowable at least after introduction of the dosage form into the operational environment. A binder, antioxidant, pharmaceutically acceptable carrier, permeation enhancer, or the like may accompany the active agent in the liquid active agent formulation 26, and the liquid active agent formulation 26 may include a surfactant of mixture of surfactants. U.S. Pat. Nos. 6,174,547 and 6,245,357 and patent publications numbered WO 95/34285, US-2002-0071863 A1, US-2003-0232078 A1, and US-2003-0198619 A1, which are incorporated herein in their entirety by reference, detail exemplary drugs, carriers, and other constituents that may be used to form a liquid active agent formulation suitable for use in the dosage form of the present invention.

An exit orifice 28 included in a dosage form 20 of the present invention may be embodied by one of various different structures suitable for allowing the release of the liquid active agent formulation 26. For example, as is shown in the figures, the exit orifice 28 included in a dosage form 20 according to the present invention may simply include an aperture 30 formed through a rate controlling membrane 24, or the exit orifice may include an aperture 30 formed through a rate controlling membrane 24 and a water impermeable subcoat 16 of dosage form 10 that includes a reservoir 22 formed of multiple material layers. An exit orifice 28 formed of an aperture 30 may be formed by any suitable means, such as by suitable mechanical or laser drilling technologies.

Though the aperture 30 illustrated in FIG. 1 through FIG. 8 does not pass entirely through the reservoir 22 included in the dosage forms 20, the aperture 30 allows the formation of an exit orifice as the dosage form is placed within or begins to operate within an intended environment of operation. In particular, where a dosage form 20 of the present invention includes a reservoir 22 formed of a single layer of water impermeable material, the aperture 30 formed in the rate controlling membrane 24 creates a breaking point where the material forming the reservoir 22 is compromised as the expandable osmotic composition 18 included in the dosage form 10 begins to function and pressure within the reservoir 22 builds. Alternatively, where a dosage form 10 of the present invention includes a water permeable material and the aperture 30 exposes such material to the environment of operation, the water present in the environment of operation can work to weaken or dissolve the exposed portion of the reservoir 20, allowing the liquid active agent formulation 26 contained within the reservoir 22 to be expelled as the permeation resistant engine 10 expands and acts against the liquid active agent formulation 26.

Nevertheless, the dosage form of the present invention is not limited to an exit orifice 28 formed by an aperture 30. Where desired, the exit orifice may include an aperture that passes completely through the rate controlling membrane and the reservoir. Again, mechanical or laser drilling technologies may be used to create such an exit orifice. However, where the exit orifice provided in the dosage form of the present invention is formed through the reservoir, a closure sealing the exit orifice be needed. Any one of several means may be employed to provide such a closure. For instance, the closure may include a layer of material that covers the exit orifice and is arranged over a portion the outer surface of the dosage form, or the closure may include a stopper, such as a bung, cork, or impermeable plug, or an erodible element, such as a gelatin plug or a pressed glucose plug, formed or positioned within the exit orifice. Regardless of its specific form, the closure will comprise a material impermeable to the passage of the liquid active agent formulation, at least until after administration of the dosage form. Suitable closure materials not already mentioned include high-density polyolefin, aluminized polyethylene, rubber, silicon, nylon, synthetic fluorine Teflon®, chlorinated hydrocarbon polyolefins, and fluorinated vinyl polymers.

An exit orifice included in a dosage form of the present invention may also include more than a simple aperture, where desired, the exit orifice may include, for example, a porous element, porous overlay, porous insert, hollow fiber, capillary tube, microporous insert, or microporous overlay. Moreover, regardless of the particular structure providing the exit orifice, a controlled release dosage form of the present invention can be manufactured with two or more exit orifices for delivering the active agent formulation during operation. Descriptions of exit orifices suitable for use in controlled release dosage forms are disclosed, for example, in those patents and patent publications already incorporated herein by reference, as well as in U.S. Pat. Nos. 3,845,770, 3,916,899, and 4,200,098, the contents of which are herein incorporated in their entirety by reference.

Though an exit orifice 28 formed of an aperture 30 is only one of various different exit orifices that may be provided in a dosage form 20 of the present invention, exit orifices 28 that are formed as shown in the illustrated embodiments are desirable, as they do not require complete penetration of the reservoir 22 before the dosage form 20 is administered. Such a design works to reduce the possibility that the liquid active agent formulation 26 may leak from the dosage form 20 before the dosage form 10 is administered. Moreover, the aperture 30 included in the exit orifices 28 shown in FIG. 1 through FIG. 8 is simply formed using known mechanical or laser drilling techniques.

Regardless of its precise configuration, the design of a dosage form according to the present invention provides a dosage form that not only provides the controlled release of liquid active agent formulations, but also better preserves the release rate functionality of the osmotic engine included in the dosage form over time and reduces the likelihood that void volume formations will occur within the reservoir of the dosage form prior to administration. Such performance is attributable to the design of the dosage form of the present invention, and, in particular, to the permeation resistant engine included in the dosage form according to the present invention. A dosage form according to the present invention may be designed to incorporate any embodiment of a permeation resistant engine according to the present invention, and in each embodiment, the dosage form of the present invention is configured to reduce or eliminate the possibility that the liquid active agent formulation included in the dosage form will come in direct contact with the expandable osmotic composition included in the permeation resistant engine.

The present invention also includes methods for fabricating a dosage form providing controlled release of a liquid active agent formulation. In each embodiment, the method of the present invention for fabricating a controlled-release dosage form includes providing a permeation resistant engine, a reservoir, and a liquid active agent formulation, loading the liquid active agent formulation into the reservoir, and operatively associating the permeation resistant engine, the reservoir and the liquid active agent formulation such that, as the permeation resistant engine operates, liquid active agent formulation is expelled from the reservoir. Any embodiment of a permeation resistant osmotic engine according to the present invention may be used in the method for fabricating a controlled release dosage form.

In one embodiment, the method of fabricating a dosage form according to the present invention includes providing a reservoir, a liquid active agent formulation, and a permeation resistant engine according to the present invention that includes an expandable osmotic composition coated with a permeation resistant coating. The reservoir is loaded with the liquid active agent formulation and the permeation resistant engine is partially inserted into the reservoir using any suitable means, such as an inserter providing insertion depth control or insertion force control. Where the permeation resistant engine is positioned within the reservoir after the liquid active agent formulation is loaded in the reservoir, it is presently preferred to insert the permeation resistant engine into the reservoir using an inserter with insertion force control, while an inserter with insertion depth control is preferred where the permeation resistant engine is positioned within the reservoir before the liquid active agent formulation is loaded therein.

In another embodiment of the method of the present invention, a dosage form is fabricated by providing a reservoir, a liquid active agent formulation, and a permeation resistant engine according to the present invention that includes a bi-layer composition formed of an expandable osmotic composition and a barrier layer. Such a bi-layer composition may be provided as a bi-layer tableted composition. Where such a permeation resistant engine is provided in a method according to the present invention, the method of the present invention includes the step of orienting the permeation resistant engine such that the barrier layer included in the permeation resistant engine is positioned between the liquid active agent formulation and the expandable osmotic composition in the finished dosage form. Again, the permeation resistant engine is preferably only partially inserted into the reservoir, and insertion of the permeation resistant engine into the reservoir may be carried out using any suitable means, such as an inserter providing insertion depth control or insertion force control.

The reservoir provided in the method of fabricating a dosage form according to the present invention may be a water impermeable reservoir or a water permeable reservoir according to the description already provided herein. However, where a method according to the present invention includes providing a water impermeable reservoir formed by a water permeable material coated with a water impermeable subcoat, the permeation resistant engine is preferably positioned within the reservoir after formation of the water impermeable subcoat. Doing so typically eases the formation of the water impermeable subcoat over the water permeable material included in the reservoir.

The method of the present invention also includes forming a rate controlling membrane. The rate controlling membrane may be formed using the methods and materials already described, and in the method of the present invention, the rate controlling membrane is formed after the permeation resistant engine and reservoir have been operatively associated. Therefore, in one embodiment, the method of fabricating a dosage form according to the present invention includes providing a reservoir, providing a permeation resistant engine, inserting the permeation resistant engine at least partially within the reservoir, and forming a rate controlling membrane over at least a portion of the reservoir or at least a portion of the permeation resistant engine such that, upon administration to an environment of operation, the permeation resistant engine expands at a controlled rate. Where the reservoir provided in a method of the present invention completely encapsulates the permeation resistant osmotic engine, the step of providing a rate controlling membrane includes providing a rate controlling membrane over at least a portion of the reservoir. However, where the method of the present invention includes providing a reservoir that does not encapsulate the permeation resistant engine and inserting the permeation resistant within the reservoir such that a portion of the permeation resistant reservoir remains exposed, the step of providing a rate controlling membrane includes providing a rate controlling membrane over at least the exposed portion of the permeation resistant engine. Of course, providing a rate controlling membrane according to the method of the present invention for fabricating a dosage form may also include providing a rate controlling membrane that substantially covers the outer surface of the reservoir or that covers both the exposed portion of the permeation resistant as well as substantially all of the outer surface of the reservoir.

The method according to the present invention for forming a dosage form also includes providing an exit orifice. The step of providing an exit orifice may include creating an aperture, or providing any other suitable device or structure that facilitates the expulsion of liquid active agent from the reservoir included in the dosage form as the permeation resistant engine functions in an environment of operation. Depending on the type of exit orifice formed, providing an exit orifice may be carried out before or after the reservoir is loaded with the liquid active agent formulation. For example, where the exit orifice includes an aperture formed through the reservoir and a plug or covering for sealing the aperture, the exit orifice will preferably be formed after the exit orifice is formed. However, where the exit orifice includes an aperture that does not completely penetrate the reservoir, the exit orifice is preferably formed after the reservoir is loaded with the liquid active agent formulation.

In each embodiment, the method according to the present invention for fabricating a controlled release dosage form facilitates the fabrication of dosage forms that provide the controlled release of liquid active agent formulations and exhibit improved long-term stability in release rate functionality and a decreased tendency to form void volumes over time. Providing a permeation resistant engine according to the present invention, in particular, allows the fabrication of a controlled-release, liquid active agent dosage form wherein migration of the liquid active agent formulation into the expandable osmotic composition included in the permeation resistant osmotic engine is reduced or eliminated altogether. Therefore, the method of the present invention for fabricating a dosage form facilitates the fabrication of a controlled-release, liquid active agent dosage form that is relatively less affected by the potential release rate inconstancies and void volume formations that may result where a dosage form is fabricated by a method that does not call for the use of a permeation resistant engine according to the present invention.

### EXAMPLES

The Examples provided below are intended to be illustrative and in no way limiting of the claimed invention.

### EXAMPLE 1

Exemplary permeation resistant osmotic engines according to the present invention were produced. The exemplary permeation resistant engines included an expandable osmotic composition and a barrier layer formed together as a bi-layer tablet. The expandable osmotic composition was formed using a standard NaCMC composition and the barrier layer was formed using Kollidon SR. The bi-layer tablet included 280 mg of the NaCMC expandable osmotic composition and 80 mg of the Kollidon barrier layer composition. To render the bi-layer tablet impermeable to a hydrophobic liquid active agent formulation and complete fabrication of the exemplary permeation resistant engines, the bi-layer tablets including the expandable osmotic composition and the barrier layer were coated with an permeation resistant coating formed of HPMC. An Aeromatic Coater was used to apply a 7% aqueous dispersion of HPMC 6 cps and PEG 8000 (90/10 w/w ratio) onto bilayer tablets under the coating conditions are described in Table 1.

### EXAMPLE 2

To quantify the uptake of hydrophobic liquid active agent formulations by the exemplary permeation resistant engines, six exemplary engines were introduced into four different liquid formulations that simulated drug formulations (Cremaphor EL, Cremaphor EL/Myvacet 50/50, Cremaphor EL/Capric Acid 75/25, and Cremaphor EL/Capric Acid 50/50). The weight gain of each engine was determined at 1 hour and approximately 50 hours post introduction into the liquid formulations.

The amount of liquid formulation absorbed by exemplary permeation resistant engines was determined by weighing each engine prior to immersion in each of the four drug formulations. The engines were removed from the liquid formulations at 1 hour, weighed a second time to determine the extent of any weight gain from absorption of liquid formulation., and then reimmersed in the liquid formulations. After approximately 50 hours, the engines were again removed from the liquid formulations and weighed a third time to again determine the extent of any weight gain from absorption of liquid active agent formulation. Each time the engines were removed from the liquid formulations for weighing, the engines were wiped thoroughly with a Kimwipe prior to weighing. The weight gain at 1 hour provided a baseline for any weight gain resulting from artifacts, such as surface roughness, that may contribute to drug formulation accumulation, rather than penetration, at the engine surface.

In order to evaluate the permeability performance provided by the exemplary permeation resistant engines, osmotic engines that did not include a permeation resistant coating (“uncoated engines”) were also prepared and immersed in the same liquid formulations. The uncoated engines were prepared exactly as the exemplary permeation resistant engines were prepared, except that the bi-layer tablets forming the uncoated engines were not coated with an HPMC permeation resistant coating. The uncoated engines were immersed in the four different drug formulations using the same protocol as was used for the exemplary permeation resistant engines. The weight gains measured for the uncoated engines served as the control.

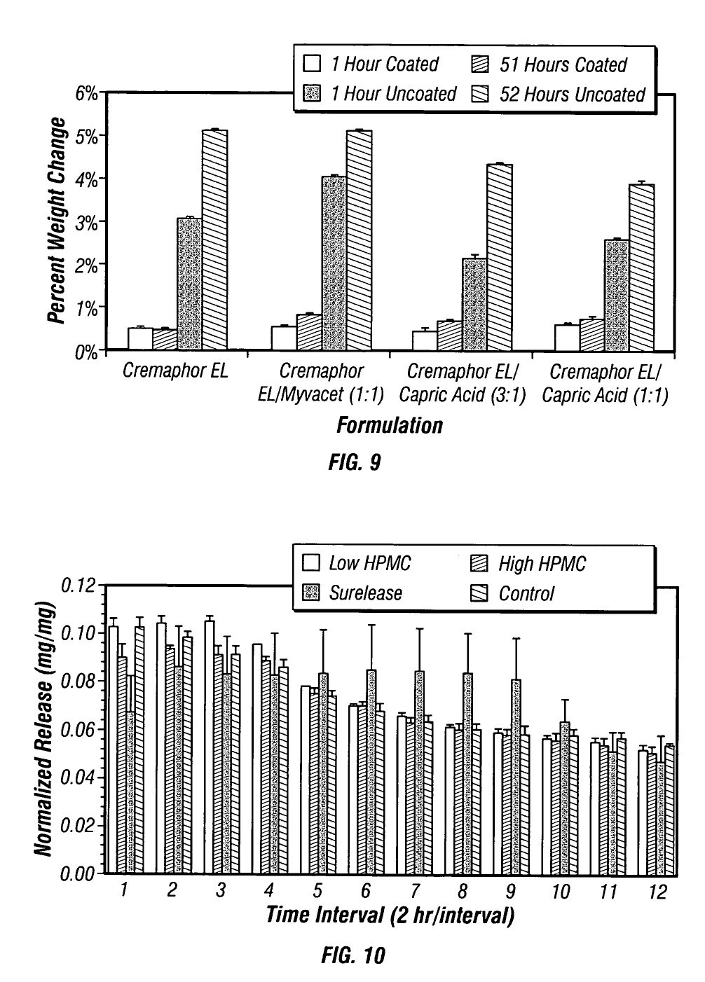

The weight gain studies revealed that the exemplary permeation resistant engines, or “coated engines,” exhibited significantly reduced uptake of liquid formulation. FIG. 9 illustrates the liquid formulation uptake for the coated and uncoated engines after 1 hour and 50 hours of immersion in each of the four liquid formulations. FIG. 9 illustrates the drug layer uptake by the coated engines (white and black bars) relative to the uncoated engines (gray and striped bars) with time. For the coated engines, the percent weight increase at 1 hour is 0.61%, 0.6%, 0.46%, and 0.59% for Cremaphor EL, Cremaphor EL/Myvacet (1:1), Cremaphor EL/Capric Acid (3:1), and Cremaphor EL/Capric Acid (1:1), respectively. After 50 hours, the coated engines showed a slight increase in the weight gain resulting from exposure to the liquid formulations, with uptakes of 0.56%, 0.87%, 0.68%, and 0.70%, respectively. The liquid formulation uptake calculated for the coated engines is relatively minor compared with the 3.12%, 4.07%, 2.16%, and 2.56% weight gains measured in the uncoated engines after 1 hour in the four respective liquid formulations. After 50 hours, the weight gains exhibited by the uncoated engines immersed in each of the liquid formulations increased to 5.16%, 5.12%, 4.31%, and 3.83%.

The relative uptake of liquid formulation for each of the engines was calculated by subtracting the 1-hour weight gains from the weight gains measured after 50 hours. Furthermore, an indicator of drug migration inhibition was introduced as the uptake inhibition factor (“UIF”). The UIF is the absolute value of the relative weight gain in the absence of coating normalized, or divided, by the relative weight gain for the exemplary permeation resistant engines, or “coated engines.” The relative uptake and UIF of each engine is listed in Table 2. High uptake inhibition factors reflect a more pronounced reduction of the drug layer intake as a result of the HPMC coating. UIF values less than 1 represent membrane coatings which promote drug migration into the osmotic engine. Based on the UIF values shown in Table 2, the presence of the HPMC coating on the osmotic engine significantly reduces migration of liquid formulation into the osmotic engine by factors of 42.21, 3.78, 9.82, and 11.64 for Cremaphor EL, Cremaphor EL/Myvacet (1:1), Cremaphor EL/Capric Acid (3:1), and Cremaphor EL/Capric Acid (1:1), respectively.

### EXAMPLE 3

The functionality of permeation resistant engines according to the present invention was evaluated. In order to conduct such an evaluation, exemplary permeation resistant engines were fabricated. The engines included a bi-layer, tableted composition as described in Example 1 and were coated with a permeation resistant HPMC coating. The exemplary engines were then used to fabricate exemplary dosage forms according to the present invention. Control dosage forms were also fabricated and the release rates of the exemplary dosage forms and the control dosage forms were evaluated.

Four different types of dosage forms were fabricated and evaluated. Each of the dosage forms included a reservoir loaded with a liquid active agent formulation formed of 5% acetaminophen in a Cremaphor EL solution. Also each reservoir included in each dosage form was provided with a 20 mil exit orifice formed by a mechanical drill. The first exemplary dosage forms included a reservoir formed of an HPMC capsule body and a permeation resistant osmotic engine inserted partially within the reservoir. The permeation resistant engine of the first exemplary dosage forms included a “high” HPMC coating over the bi-layer tableted composition. The high HPMC coating was approximately 18.6 mg and 3.4 mils thick. The second exemplary dosage forms were manufactured as were the first exemplary dosage forms, except that the permeation resistant engines included in the second exemplary dosage forms included a bi-layer tableted composition was coated by a “low” HPMC coating. The low HPMC coating was approximately 5.6 mg and 0.92 mils thick. The first control dosage forms were fabricated exactly as the first and second exemplary dosage forms were fabricated, except that the osmotic engine included in the control dosage forms did not include a permeation resistant coating. The second control dosage forms were manufactured just as the first control dosage forms, except that reservoir used in the second control dosage forms was formed using an HPMC capsule body coated by a water impermeable subcoat. The water impermeable subcoat included in the reservoirs of the second control dosage forms was formed by coating the HPMC capsule bodies included in the reservoirs with a Surelease coating (about 62 mg). All of the dosage forms evaluated were provided with a rate controlling membrane that coated both the portion of the osmotic engines left exposed by the reservoir and the reservoir itself. Table 3 lists the coating conditions used for to provide the dosage forms with a rate controlling membrane. The release of the liquid active agent formulation from each of the dosage forms evaluated was measured over 24 hours at 2 hour intervals, and the release rate experiments were performed in triplicate.

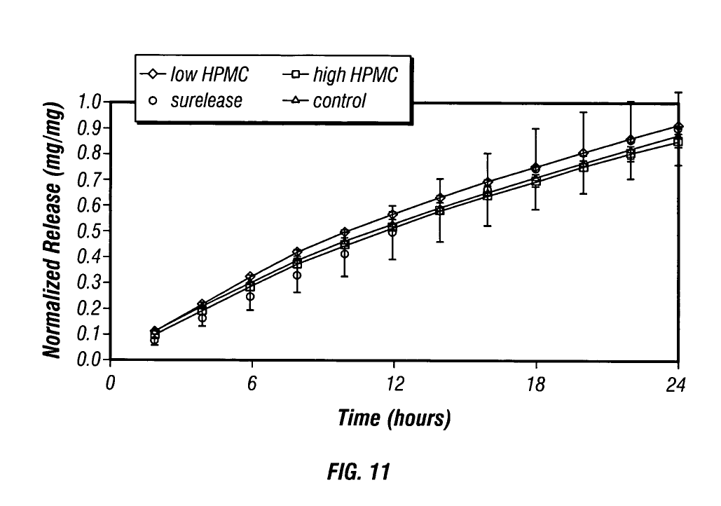

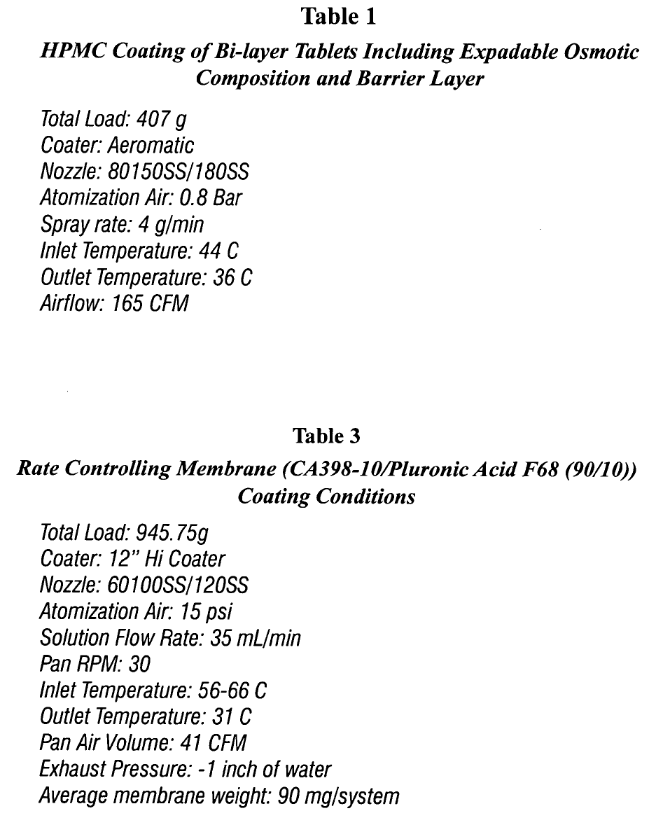

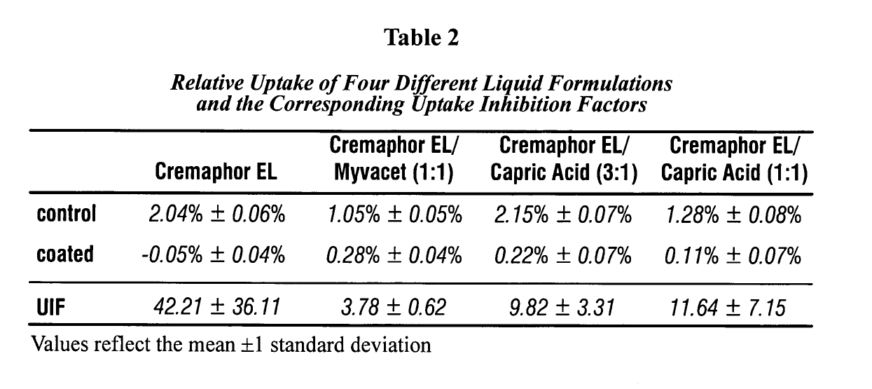

The release rate functionality for the four different dosage forms is shown in FIG. 10 and FIG. 11. The error bars indicate 1 standard deviation from the mean. Comparing the release rates between the exemplary dosage forms incorporating the low and the high HPMC-coated engines (compare the gray bars with the white bars), the high HPMC coated engines exhibited a slower release over the first 8 hours. The average difference between these two normalized release rates over the first eight hours was 0.011 mg/total mg per 2 hour interval. This difference in release rate profile indicates that the configuration (e.g., the weight) of a permeation resistant coating included in a permeation resistant engine according to the present invention may be utilized as an additional parameter that enables the establishment of drug-specific release rate profiles. Following the first eight hours, the release rates provided by the two different exemplary dosage forms approached a difference of merely 0.001 mg/total mg per 2 hour interval. Both release rates followed a decreasing release profile.

Relative to the release rate performance of the first control dosage forms, which did not include a water impermeable reservoir, the exemplary dosage forms exhibited a steadier zero-order profile during the first eight hours, prior to the decline in release rates. The initial release after 2 hours for the first control dosage forms was comparable to the release rate provided by the exemplary dosage forms including the permeation resistant engines with the low HPMC coating. The release rate of the first control dosage form, however, consecutively decreases over the next time intervals (see FIG. 10, black bars) to approach the release rates achieved by both of the different exemplary dosage forms in intervals 6 through 12. Comparison of the release rate functionality of the first control dosage forms and the release rate functionality provided by first and second exemplary dosage forms, indicates that the permeation resistant osmotic engines incorporated in the first and second exemplary dosage forms did not hinder engine or dosage form performance. In fact, based on the results presented above, the permeation resistant engines served to stabilize the zero order release rate at the outset of the dosage form operation.

The second control dosage forms maintained a zero order release profile from interval 2 through 9, or over 16 hours, despite the higher variability captured by the relative standard deviations that range from 16% to 25% (see error bars corresponding to the striped bars in FIG. 10). Compared to the first control dosage forms and the exemplary dosage forms, the start-up time of the second control dosage forms was slower, with a normalized release rate of 0.068 mg/total mg per 2 hour interval.

1. An osmotic engine suitable for use in dosage forms providing controlled delivery of active agent formulations comprising:
a permeation resistant osmotic engine that is resistant to permeation by liquid active agent formulations.

2. The osmotic engine of claim 1, wherein the permeation resistant osmotic engine comprises an expandable osmotic composition with a permeation resistant coating provided over at least a portion of the expandable osmotic composition.

3. The osmotic engine of claim 2, wherein the permeation resistant osmotic engine comprises an expandable osmotic composition encapsulated by a permeation resistant coating.

4. The osmotic engine of claim.1, further comprising a barrier layer.

5. The osmotic engine of claim 1, further comprising a barrier layer provided within the permeation resistant coating or outside of the permeation resistant coating.

6. The osmotic engine of claim 2, wherein the permeation resistant coating comprises a hydrophobic coating formulated to reduce or prevent permeation by an aqueous or hydrophilic liquid active agent formulation.

7. The osmotic engine of claim 2, wherein the permeation resistant coating comprises a hydrophilic coating formulated to reduce or prevent permeation by a hydrophobic liquid active agent formulation.

8. The osmotic engine of claim 2, wherein the permeation resistant coating comprises a permeation resistant coating formulated or configured to allow the passage of water from an environment of operation into the expandable osmotic composition.

9. A method for manufacturing a permeation resistant osmotic engine comprising
providing an expandable osmotic composition,
coating said expandable osmotic composition with a permeation resistant coating that covers at least a part of an outside surface of the expandable osmotic composition.

10. The method of claim 9, further comprising
substantially encapsulating the expandable osmotic composition in a permeation resistant coating.

11. The method of claim 9 further comprising
providing an expandable osmotic composition and a barrier layer,
providing a permeation resistant coating over an outside surface of the barrier layer and over at least a portion of an outside surface of the expandable osmotic composition.

12. The method of claim 9, wherein the expandable osmotic composition and barrier layer are provided as a bi-layer tableted composition.

13. A method for fabricating a permeation resistant engine comprising
providing an expandable osmotic composition,
coating said composition with a permeation resistant coating that at least partially covers an outside surface of the expandable osmotic composition, and
positioning a barrier layer over an outside surface of the permeation resistant coating.

14. The method of claim 13, wherein the barrier layer comprises a barrier layer adhered to or positioned in contact with the permeation resistant coating.

15. An osmotic dosage form that provides controlled delivery of a liquid active agent formulation comprising:
a permeation resistant osmotic engine
a reservoir, and
a liquid active agent formulation contained within the reservoir.

16. The osmotic dosage form of claim 15, wherein the permeation resistant osmotic engine is configured to reduce or prevent migration of the liquid active agent formulation into an expandable osmotic composition of which the permeation resistant osmotic engine is comprised.

17. An osmotic dosage form comprising:
a reservoir,
a liquid active agent formulation within the reservoir,
a permeation resistant osmotic engine that comprises an expandable osmotic composition and a permeation resistant coating,
a rate controlling membrane, and
an exit orifice through which the liquid active agent formulation can be delivered.

18. A method for fabricating a dosage form providing controlled release of a liquid active agent formulation comprising:
providing a permeation resistant engine, a reservoir, and a liquid active agent formulation,
loading the liquid active agent formulation into the reservoir, and
operatively associating the permeation resistant engine, the reservoir and the liquid active agent formulation such that, as the permeation resistant engine operates, liquid active agent formulation is expelled from the reservoir.

19. The method of claim 18, further comprising
providing a rate controlling membrane that is formulated and configured to provide controlled expansion of the permeation resistant engine upon administration of the dosage form to an environment of operation, and
providing an exit orifice that allows the liquid active agent formulation to be expelled from within the reservoir as the dosage form operates.
